(19) United States
(12) Patent Application Publication
(10) Pub. No.: US 2020/0268634 A1
(43) Pub. Date: Aug. 27, 2020

(54) EYE MAKE-UP COSMETIC COMPOSITION COMPRISING ACRYLATE-BASED COPOLYMER

(71) Applicant: COSMAX, INC., Hwaseong (KR)

(72) Inventors: Mi Na LEE, Osan (KR); Seok Won CHOI, Hwaseong (KR); Myeong Sam PARK, Seoul (KR)

(73) Assignee: COSMAX, INC., Hwaseong (KR)

(21) Appl. No.: 16/652,357

(22) PCT Filed: Jan. 3, 2018
(86) PCT No.: PCT/KR2018/000097

§ 371 Date: Mar. 30, 2020

(30) Foreign Application Priority Data
Oct. 13, 2017 (KR) 10-2017-0133278

(51) Int. Cl. A61K 8/81; A61K 8/02; A61Q 1/10

(52) U.S. Cl. CPC A61K 8/8147; A61K 2800/412; A61Q 1/10; A61K 8/0241

(57) ABSTRACT

The present invention provides an eye make-up cosmetic composition comprising an acrylate-based copolymer, a preparation method therefor, and a cosmetic product using the same.

### TECHNICAL FIELD

The present invention relates to an eye make-up cosmetic composition.

### BACKGROUND ART

Eye make-up cosmetic products include, for example, shadows, liners, mascaras, etc.

Eye shadow make-up compositions are greatly divided into powder types, cream types, liquid types, etc. depending on the formulation. Liners are divided into liquid liners, brush pen liners, pencil liners, and gel eye liners depending on the container and formulation. Mascaras are divided into volume, curling, and lash depending on the cosmetic effects, and are also divided into fixers, browcaras, etc. depending on the use.

In order to make eye liner compositions having high blackness, in general, carbon black colorants are used. However, carbon black is highly glossy. So, in order to make eye liner compositions which are not so glossy, silica particles are mainly used to lower the gloss. However, among the eye make-up products, the brush pen liners have a problem that silica particles having a great particle size are not dispensed.

Accordingly, it is necessary to develop eye make-up cosmetic compositions which have low gloss and thus are capable of producing a natural eye make-up.

### DETAILED DESCRIPTION OF INVENTION

### Technical Task

The present invention aims to provide an eye make-up cosmetic composition with low gloss cosmetic effects.

### Means for Solving the Task

One embodiment of the present invention provides an eye make-up cosmetic composition which comprises an acrylate-based copolymer having a glass transition temperature of 80-120° C.

The cosmetic composition of the present invention may comprise 2-25% by weight of acrylate-based copolymer.

The acrylate-based copolymer may be at least one selected from the group consisting of styrene acrylate copolymers, styrene/acrylate/ammonium methacrylate copolymers, sodium styrene/acrylate copolymers, ammonium acrylate copolymers, ammonium VA/acrylate copolymers, sodium acrylate copolymers, ethylene/acrylic acid copolymers, ethylene/calcium acrylate copolymers, ethylene/magnesium acrylate copolymers, ethylene/sodium acrylate copolymers, ethylene/zinc acrylate copolymers, ethylene/acrylic acid/VA copolymers, acrylate/VP copolymers, acrylate/VA copolymers, steareth-10 aryl ether/acrylate copolymers, acrylate/steareth-50 acrylate copolymers, acrylate/steareth-20 methacrylate copolymers, acrylate/ammonium methacrylate copolymers, ammonium styrene/acrylate copolymers, acrylate/hydroxyl ester acrylate copolymers, methacryloyl ethyl betaine/acrylate copolymers, lauryl acrylate/VA copolymers, VA/butyl maleate/isobornyl acrylate copolymers, ethylene/methacrylate copolymers, vinyl caprolactam/VP/dimethyl amino ethyl methacrylate copolymers, sodium acrylate/acrolein copolymers, VP/dimethyl amino ethyl methacrylate copolymers, and AMP-acrylate copolymers.

The acrylate copolymer may have an average particle size of 0.5-3 μm.

Additionally, the eye make-up cosmetic composition of the present invention may further comprise at least one selected from the group consisting of polyol, preservatives, surfactants, oil, wax, colorant and purified water.

Another embodiment of the present invention provides an eye make-up cosmetic product comprising the cosmetic composition. Preferably, the cosmetic product is a brush pen eye liner.

Yet another embodiment of the present invention provides a method of preparing an eye make-up cosmetic composition comprising the steps of a) mixing colorants in an aqueous phase part and stirring the same; and b) mixing an acrylate-based copolymer as a film forming agent in the aqueous phase part and stirring the same.

The aqueous phase part in step a) may comprise at least one selected from the group consisting of polyol, preservatives, surfactants, and purified water.

### Effect of Invention

The present invention relates to an eye make-up cosmetic composition comprising an acrylate-based copolymer, capable of implementing cosmetic effects which are not so glossy and thus are matt.

Furthermore, the cosmetic composition of the present invention can be easily dispensed from any containers, so it can be applied to various formulations.

### BRIEF DESCRIPTION OF DRAWINGS

FIG. 1 is a photograph taken after applying a cosmetic composition of a example 3 and a comparative example 1 on the back of the hand; and

FIG. 2 is an enlarged photograph taken using an Aram huvis APM instrument after applying a cosmetic composition of a example 3 and a comparative example 3 on the back of the hand.

### BEST MODES FOR CARRYING OUT THE INVENTION

One embodiment of the present invention provides an eye make-up cosmetic composition which comprises an acrylate-based copolymer having a glass transition temperature of 80 to 120° C.

The cosmetic composition of the present invention may comprise 2-25% by weight, preferably 2-20% by weight, more preferably 2-15% by weight, and most preferably 3-10% by weight of the acrylate-based copolymer. When the content of the copolymer is less than 2% by weight, the gloss-lowering effects are insignificant, and when the content of the copolymer exceeds 25% by weight, the feeling of use or stability would be reduced, which is not preferable.

The acrylate copolymer may have an average particle size of 0.5-3 μm, preferably 0.7-2.5 μm, and more preferably 1-2 μm. When the average particle size of the copolymer is less than 0.5 μm, the gloss lowering effects are insignificant, and when the average particle size of the copolymer exceeds 3 μm, since the size of the particle is great, the user would not have a good feeling of use in terms of cosmetic effects, which is not preferable.

The acrylate-based copolymer may have a glass transition temperature of 80-120° C., 85-120° C., 90-120° C., 95-120° C., 100-120° C., 105-120° C., 110-120° C., 80-110° C., 85-110° C., 90-110° C., 95-110° C., 100-110° C., 80-105° C., 85-105° C., 90-105° C., 95-105° C., 100-105° C., 80-100° C., 85-100° C., 90-100° C., and 95-100° C., but is not limited thereto. When the glass transition temperature of the acrylate-based copolymer is less than 80° C., the glossiness of the eye make-up cosmetic composition may increase and the transparency may become high when dried after use, thereby reducing matt effects. When the temperature exceeds 120° C., the cosmetic composition may cover the skin in white while being dried when applied to the skin, which is not preferable.

Also, the eye make-up cosmetic composition of the present invention may further comprise at least one selected from the group consisting of polyol, preservatives, surfactants, oil, wax, colorant and purified water.

According to the present invention, the polyol is a high molecular compound having a hydroxyl group which, for example, may be at least one selected from the group consisting of ethylene glycol, propylene glycol, glycerin, butylene glycol, and a combination thereof, but is not limited thereto.

The preservatives refer to all raw materials which melt in an oil phase and represent preservative activity, and may include, as a natural raw material, an essential oil extracted by a steam distillation method having anti-bacterial and anti-inflammatory effects. As an example, organic acids serving as preservatives [acetic acid, butyric acid, citric acid, palmitic acid, oxalic acid, tartaric acid, glycolic acid, lactic acid, maleic acid], etc. such as phenoxyethanol, ethanol, and pentylene glycol, hexanediol, ethylhexylglycerin, caprylyl glycol, glycerin, butylene glycol, propylene glycol, etc., which are polyalcohols, may be used.

Furthermore, the surfactants include anionic surfactants, cationic surfactants, amphoteric surfactants, and nonionic surfactants, and refer to a concept which includes all ingredients serving as emulsifying agents, dispersing agents, and solubilizing agents. Specifically, the surfactants may include at least one selected from glyceryl stearate, polysorbate 20, potassium cetyl phosphate, and lecithin.

The oil, for example, may be natural vegetable oil, but is not particularly limited thereto. The natural vegetable oil refers to oil extracted from plant-derived raw materials by using extraction methods such as a supercritical extraction method, a steam extraction method, a physical extraction method, etc., but is not limited to specific examples. For example, the oil may be at least one selected from the group consisting of argan oil, jojoba oil, sunflower seed oil, sweet almond oil, immortelle oil, clove oil, patchouli oil, carrot seed oil, sunflower seed oil, castor oil, grape seed oil, soybean oil, rose hip oil, macadamia nut oil, rice bran oil, and olive oil.

The wax refers to a higher monohydric or dihydric alcohol fatty acid ester which does not melt in water, that is, an ingredient handling the hardness of lipid components. The wax of the present invention refers to any physiologically acceptable material, specifically, any cosmetically acceptable material, and particularly may be derived from mineral, animal nature, plants or a combination thereof. For example, the wax may include at least one selected from the group consisting of beeswax, canderilla wax, rice bran wax, cotton plant wax, carnauba wax, esparto wax, Japan wax, Montan wax, sugar cane wax, bee wax, wool wax, shellac, micro wax, ceresin, ozokerite, ouricuri wax, cork fiber wax, brown coal wax, berry wax, shea butter or synthetic wax, for example, paraffin wax, polyethylene wax, wax prepared by Fischer-Tropsche synthesis, hydrogenated oil, silicone wax and derivatives thereof (alkyl derivatives, alkoxy derivatives, and/or ester of polymethylsiloxane).

The colorant may include at least one selected from the group consisting of organic colorant, inorganic colorant, animal and vegetable natural colorant, white pigment, dye, inorganic pigment, organic pigment, lake colorant, pearl, and other coloring pigments.

Additionally, as one example, the eye make-up cosmetic composition may comprise 0.5-30% by weight of polyol, 0.5-10% by weight of preservatives, 0.5-10% by weight of surfactants, 1-50% by weight of oil, 1-50% by weight of wax, 1-50% by weight of colorant, and 1-90% by weight of purified water.

Another embodiment of the present invention provides an eye make-up cosmetic product comprising the cosmetic composition. Preferably, the cosmetic product is a brush pen eye liner. Since the present invention uses a copolymer having an average particle size of 0.5-3 μm as a film forming agent, the copolymer can be dispensed even in brush pen eye liners. Also, the copolymer may be applied to various formulations other than brush pen eye liners.

Yet another embodiment of the present invention provides a method for preparing the eye make-up cosmetic composition comprising the steps of a) mixing colorants in an aqueous phase part and stirring the same; and b) mixing an acrylate-based copolymer as a film forming agent in the aqueous phase part and stirring the same.

The aqueous phase part in the step a) may comprise at least one selected from the group consisting of polyol, preservatives, surfactants, oil, wax and purified water.

More specifically, before step a), the step of stirring the aqueous phase part comprising polyol, preservatives, surfactants, oil, wax and purified water may be further carried out.

### MODES FOR CARRYING OUT THE INVENTION

### Preparation in Examples and Comparative Examples

In examples 1-3 and comparative examples 1-2, each raw material was measured in beakers according to the ingredient and content indicated in table 1 below, and was uniformly mixed at room temperature using a disper. After inserting polyol, dispersing agents, and purified water, the mixture was uniformly mixed for 5 minutes at 300 rpm. Next, the colorant was inserted and the mixture was uniformly mixed for 10 minutes at 500 rpm. Then, an acrylate-based copolymer (styrene acrylate copolymer, average particle size 1-2 μm, Tg=95-110° was inserted as a film forming agent, and the mixture was uniformly mixed for 30 minutes at 800 rpm. After removing bubbles, each cosmetic composition was prepared.

Table 1 below shows the ingredient and content of the cosmetic compositions of examples 1-3 and comparative example 1.

TABLE 1

| | Ingredients (content | | | | Comparative | Comparative |
|---|---|---|---|---|---|---|
| | unit: % by weight) | Example 1 | Example 2 | Example 3 | example 1 | example 2 |
| 1 | Purified water | to 100 | to 100 | to 100 | to 100 | to 100 |
| 2 | Polyol | 5 | 5 | 5 | 5 | 5 |
| 3 | Preservative | suitable amount | suitable amount | suitable amount | suitable amount | suitable amount |
| 4 | Colorant | suitable amount | suitable amount | suitable amount | suitable amount | suitable amount |
| 5 | Acrylate-based copolymer | 2.00 | 3.00 | 5.00 | — | 1.00 |

### Experimental Example 1. Evaluation of Glossiness

As a result of the preparation experiments, a composition having low gloss was obtained in example 1-3, whereas a composition having high gloss was obtained in comparative example 1. Comparative example 2 had a glossiness similar to comparative example 1 when compared with the naked eye.

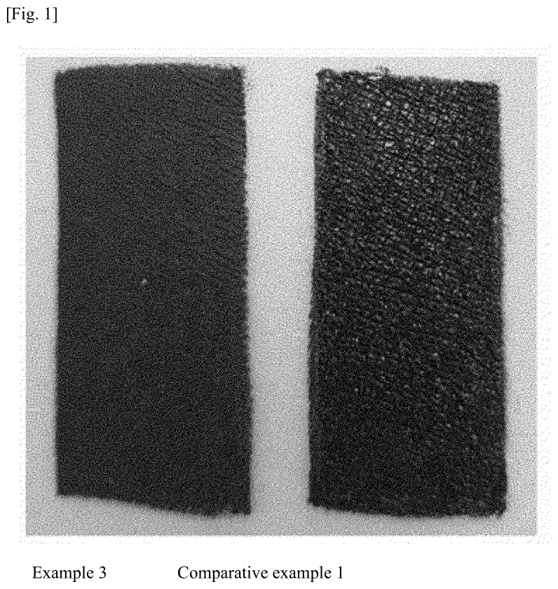

FIG. 1 is a photograph in which the cosmetic composition prepared in comparative example 1 and example 3 is applied on the back of the hand. It can be confirmed even from the naked eye that the cosmetic composition of the present invention has very low gloss.

Next, in order to compare the glossiness when comprising acrylate-based copolymers having different particle sizes and Tg values with respect to the content of 5.00% by weight, which exhibits low gloss cosmetic effects, based on the examples in Table 1, cosmetic compositions of the ingredients and contents as shown in Table 2 below were prepared.

TABLE 2

| | Ingredient (content | | Comparative |
|---|---|---|---|
| | unit: % by weight) | Example 3 | example 3 |
| 1 | Purified water | to 100 | to 100 |
| 2 | Polyol | 5 | 5 |
| 3 | Preservative | suitable amount | suitable amount |
| 4 | colorant | suitable amount | suitable amount |
| | Styrene acrylate | 5.00 | — |
| | copolymer (average | | |
| | particle size 1-2 μm, | | |
| | Tg = 95-110° C.) | | |
| 6 | Styrene acrylate | — | 5.00 |
| | copolymer (average | | |
| | particle size 0.125 μm, | | |
| | Tg = 7° C.) | | |

The cosmetic compositions in comparative example 3 and example 3 were applied to the arm by 3 cm*1 cm, respectively, and then were dried for 10 minutes. Thereafter the glossiness was measured by the BYK Gardner glossmeter five times each, and the results are shown in Table 3 below.

TABLE 3

| | 1 | 2 | 3 | 4 | 5 |
|---|---|---|---|---|---|
| Comparative | 1.00 | 1.20 | 1.10 | 1.10 | 1.10 |
| example 3 | | | | | |
| Example 3 | 0.50 | 0.60 | 0.50 | 0.60 | 0.60 |

According to the result, comparative example 3 has a glossiness ranging between 1.00 and 1.20 gloss unit, and example 3 has a glossiness ranging between 0.50 and 0.60 gloss unit, which is lower by about 40%. For reference, the glossiness of the skin is 1.7.

### Experimental Example 2. Evaluation of Feeling of Use and Cosmetic Effects

An experiment was carried out to evaluate the feeling of use of the compositions prepared in examples 1-3 and comparative examples 2-3.

In order to evaluate the feeling of use, 20 women aging between 20 and 30 were asked to compare the effect of the products in examples 1-3 and comparative examples 2-3. Each item was evaluated and scored from 0 to 5, and the scores were averaged. A result thereof is shown in Table 4 below.

<Evaluation Standard for Uniform Line Formation>

1: very ununiform 2: ununiform 3: average 4: uniform 5: very uniform

TABLE 4

| | | | | Compar- | Compar- |
|---|---|---|---|---|---|
| | | | | ative | ative |
| Test item | Example 1 | Example 2 | Example 3 | example 2 | example 3 |
| Uniform line | 4.7 | 4.8 | 5.0 | 3.5 | 2.1 |
| formation | | | | | |

As shown in Table 4 above, there is almost no blank portion between the lines in examples 1-3, so no rough feeling is made, and thus cosmetic effects of providing eye lines with no blank portions may be achieved.

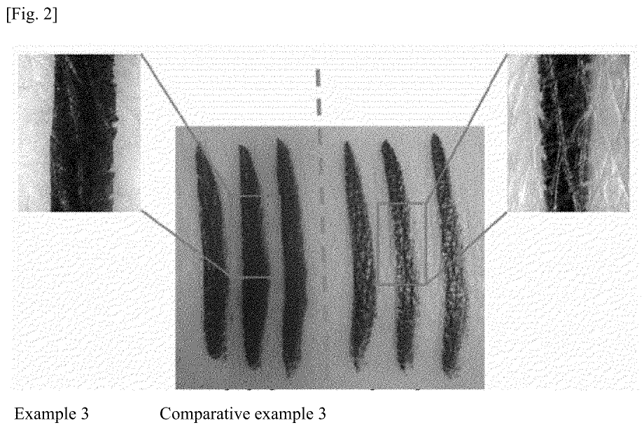

Also, FIG. 2 is a photograph in which the cosmetic composition prepared in comparative example 3 and example 3 is applied on the back of the hand. Since example 3 of the present invention forms eye lines with no blank portions, excellent cosmetic effects may be implemented.

1. An eye make-up cosmetic composition, comprising an acrylate-based copolymer having a glass transition temperature of 80-120° C.

2. The eye make-up cosmetic composition according to claim 1, comprising 2-25% by weight of acrylate-based copolymer.

3. The eye make-up cosmetic composition according to claim 1, wherein the acrylate-based copolymer is at least one selected from the group consisting of styrene acrylate copolymers, styrene/acrylate/ammonium methacrylate copolymers, sodium styrene/acrylate copolymers, ammonium acrylate copolymers, ammonium VA/acrylate copolymers, sodium acrylate copolymers, ethylene/acrylic acid copolymers, ethylene/calcium acrylate copolymers, ethylene/magnesium acrylate copolymers, ethylene/sodium acrylate copolymers, ethylene/zinc acrylate copolymers, ethylene/acrylic acid/VA copolymers, acrylate/VP copolymers, acrylate/VA copolymers, steareth-10 aryl ether/acrylate copolymers, acrylate/steareth-50 acrylate copolymers, acrylate/steareth-20 methacrylate copolymers, acrylate/ammonium methacrylate copolymers, ammonium styrene/acrylate copolymers, acrylate/hydroxyl ester acrylate copolymers, methacryloyl ethyl betaine/acrylate copolymers, lauryl acrylate/VA copolymers, VA/butyl maleate/isobornyl acrylate copolymers, ethylene/methacrylate copolymers, vinyl caprolactam/VP/dimethyl amino ethyl methacrylate copolymers, sodium acrylate/acrolein copolymers, VP/dimethyl amino ethyl methacrylate copolymers, and AMP-acrylate copolymers.

4. The eye make-up cosmetic composition according to claim 1, wherein the acrylate-based copolymer has an average particle size of 0.5-3 μm.

5. The eye make-up cosmetic composition according to claim 1, further comprising at least one selected from the group consisting of polyol, preservatives, surfactants, oil, wax, colorant and purified water.

6. An eye make-up cosmetic product, comprising the cosmetic composition according to claim 1.

7. A method of preparing an eye make-up cosmetic composition, comprising:
a) mixing colorants in an aqueous phase part and stirring the same; and
b) mixing an acrylate-based copolymer as a film forming agent in the aqueous phase part and stirring the same.

8. The method according to claim 7, wherein the aqueous phase part in step a) comprises at least one selected from the group consisting of polyol, preservatives, surfactants, oil, wax and purified water.
